(19) United States
(12) Patent Application Publication
(10) Pub. No.: US 2012/0150166 A1
(43) Pub. Date: Jun. 14, 2012

(54) Ultrashort Pulse Laser Subsurface Tissue Modifications

(76) Inventor: Joseph Neev, Laguna Beach, CA (US)

(21) Appl. No.: 13/248,001

(22) Filed: Sep. 28, 2011

Related U.S. Application Data
(60) Provisional application No. 61/387,010, filed on Sep. 28, 2010.

(51) Int. Cl. A61B 18/18; A61N 1/00

(52) U.S. Cl. 606/33; 607/2; 606/43

(57) ABSTRACT

For Example, said method and device are directed towards the treatment of skin and subsurface structure of skin for removal of hair, skin protection from Sun light and other externally damaging effects, bacteria depositions, and reduction of hair, acne, sweat, wrinkles among other applications in the skin. Additional example of embodiments of the present invention are subsurface and surface treatment of the cornea, crystalline lens, retina and other ophthalmology applications in treatment of the eye.

### RELATED APPLICATIONS

This application claims priority to U.S. Provisional Application Ser. No. 61/387,010 titled “ULTRASHORT PULSE LASER SUBSURFACE TISSUE MODIFICATION”, filed on Sep. 28, 2010, all of which are hereby incorporated herein by reference in their entireties.

This application claim priority from Provisional Patent Application No. 61/387,010 filed on Sep. 28, 2010 and titled: Ultrashort Pulse Laser Subsurface Tissue Modification The entire content of which is hereby incorporated by reference, in its entirety.

### BACKGROUND

The invention describes a device and a method for interaction with tissue underneath the surface of a mammal body, for example, underneath the surface of a human body.

It is known in the art to image subsurface mammal tissue underneath the surface with ultrasound.

It is known in the art to image subsurface mammal tissue underneath the surface with MRI, Functional MRI, CT, X-Ray equipment, and gamma rays, beta radiation, and proton beams.

Some of these devices and energy sources used for imaging can also be directed towards subsurface body components and create a tissue-modifying interaction that ablate, coagulate or otherwise modify the subsurface targeted tissue.

For example, a proton beams are used to ablate or otherwise destroy tumors in the eye.

A major deficiency of such Prior Art 3D Tissue Modification and Imaging Methods is the lack of precision (or depth resolution) and control over collateral damage. For example, ultrasound energy can be used for imaging and targeting of tumors but its resolution is limited to the order of about a millimeters. High frequency ultrasound microscopy can be used to achieve higher resolution (e.g. to of hundreds of micrometer), but such an improved resolution severely curtails the ultrasound energy ability to penetrate the tissue and is, therefore, used mainly in ultrasound microscopy.

Another serious and dangerous limitation of the prior art three dimensional tissue modification methods is that some of the energy sources is the fact that some of them are ionizing energy sources and thus can cause cancer in addition to the non-target specific collateral damage they produce.

In another prior art known to those skilled in the art optical coherent tomography is used to image at least some of the targeted tissue. This method is limited by the optical penetration of the light and scattering of the light by the targeted medium and the overlying layers.

The device and method described herein overcome these limitations and offer a novel subsurface tissue targeting and imaging technologies.

### BRIEF DESCRIPTION OF PREFERRED EMBODIMENTS

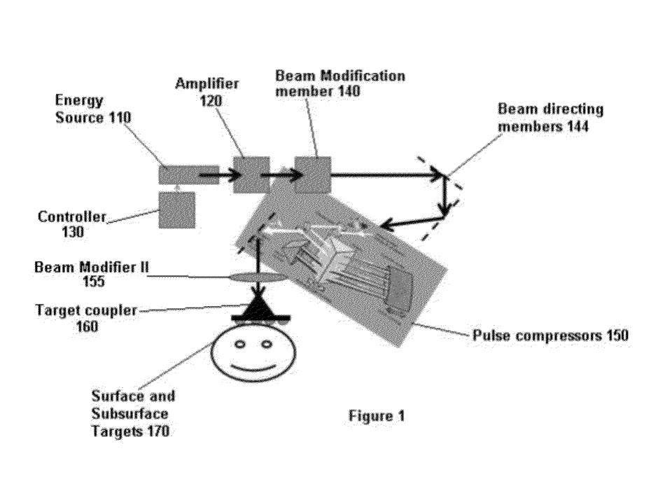

FIG. 1 Shows a device for treating subsurface target, for example in the brain, with pulse compression and tissue compression.

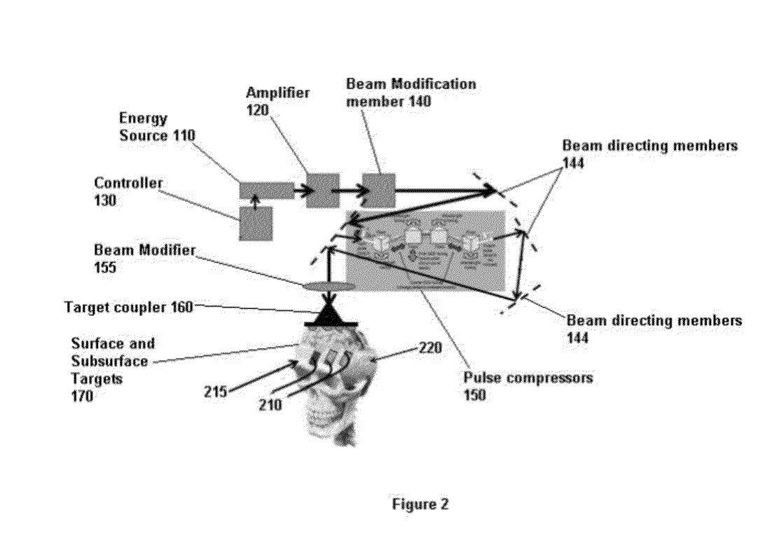

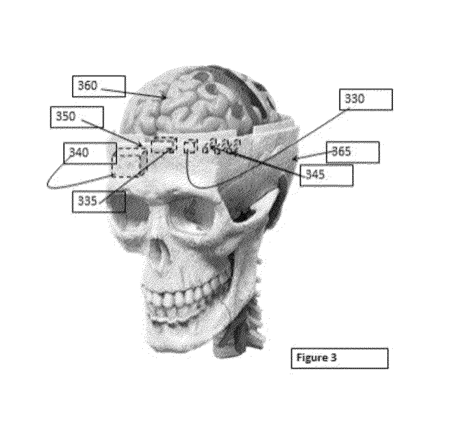

FIG. 2 shows further embodiment of a device and a method for treating subsurface target, for example in the brain, with pulse compression and tissue compression.

FIG. 3 shows further embodiment for targets of treatment in the skull and brain.

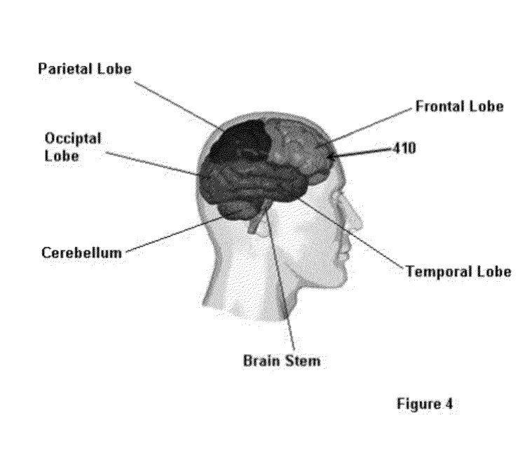

FIG. 4 shows another embodiment for targets of treatment in the skull and brain

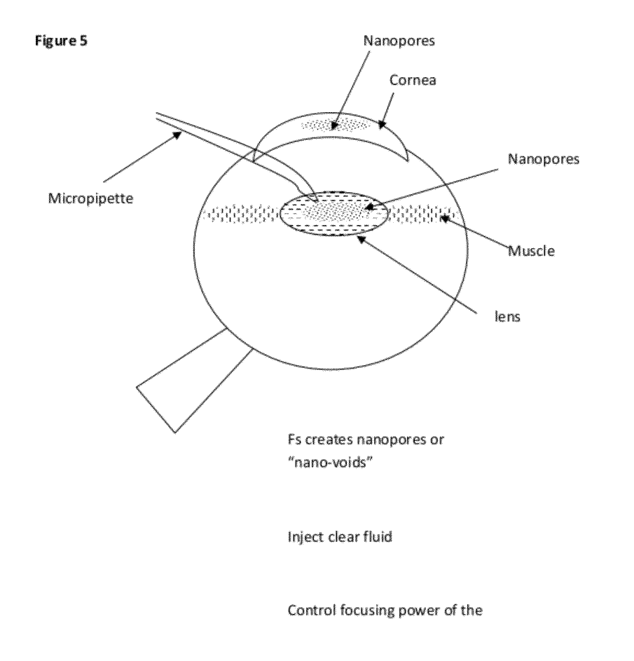

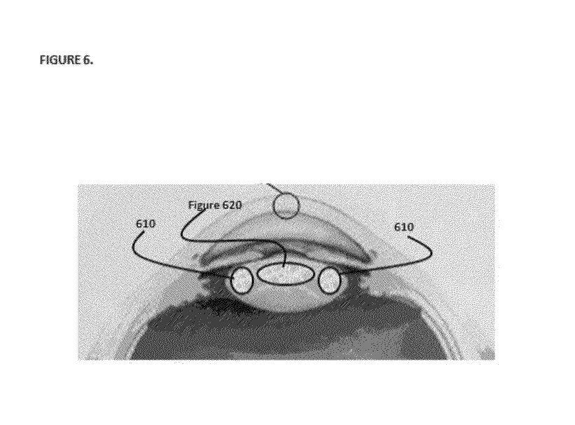

FIG. 5 shows an embodiment for treating targets and ailment of the eye.

FIG. 6 shows further embodiment for treating targets and ailment of the eye.

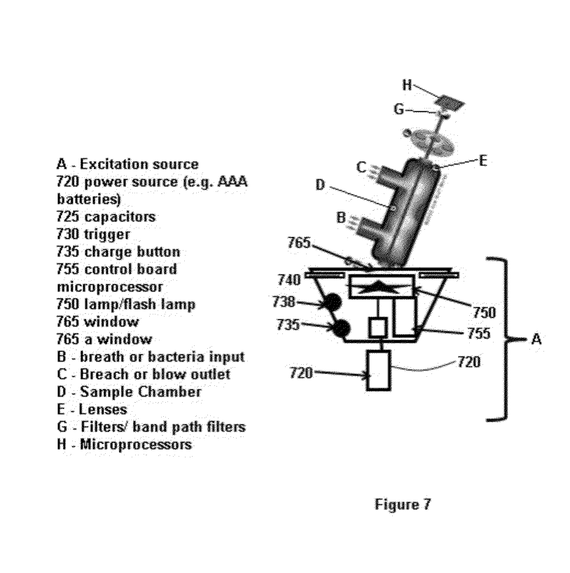

FIG. 7 shows an embodiment of the present invention to detect bacteria or chemical components.

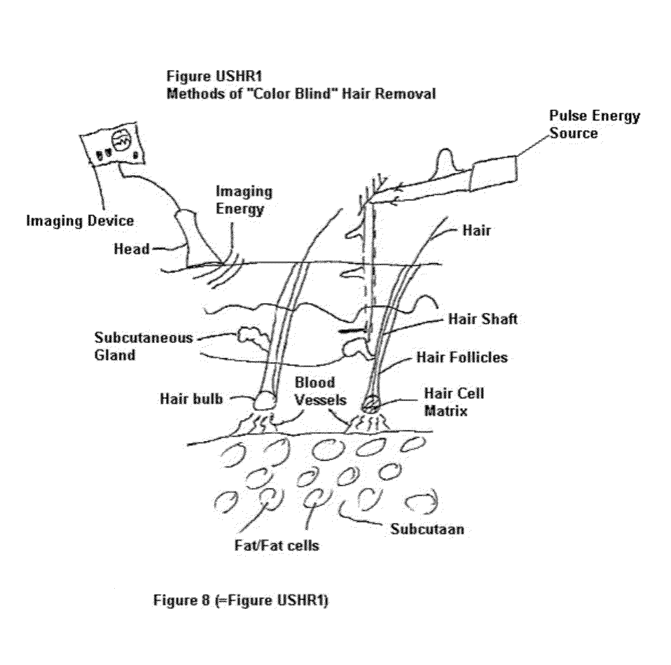

FIG. 8 shows further embodiment of a device and a method for treating subsurface targets, for example in hair follicles in the skin, with pulse compression and tissue compression.

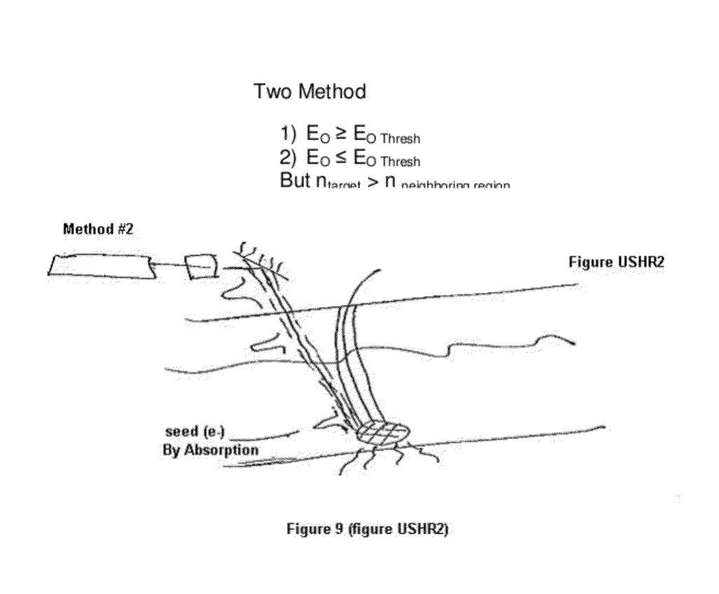

FIG. 9 shows further embodiment of a device and a method for treating subsurface target, for example in the skin, for example, hair follicle using two different methods of the present invention.

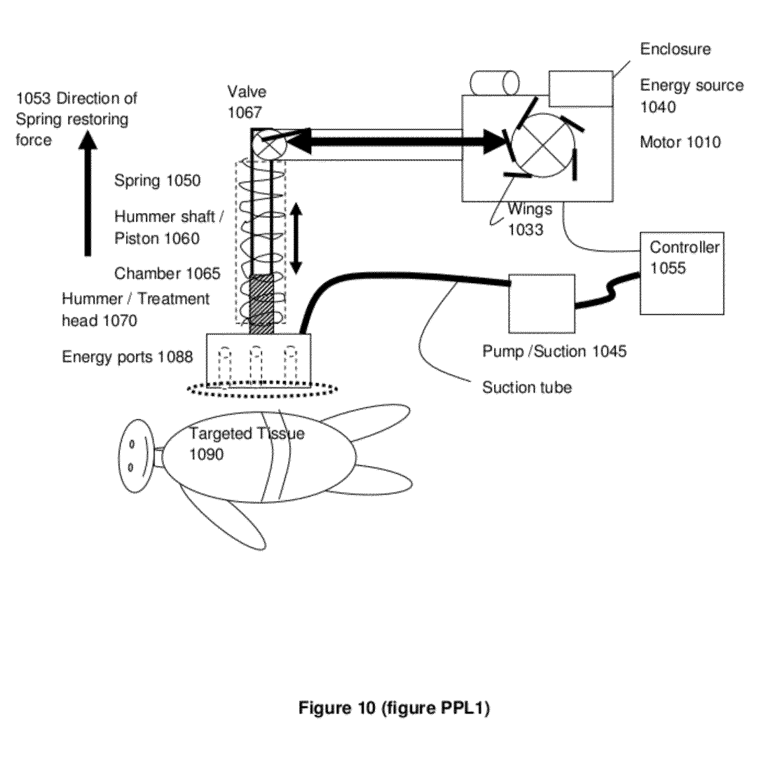

FIG. 10 shows an embodiment of a device for periodic pulsed Electromagnetic energy and periodic pulsed mechanical energy treatment of tissue.

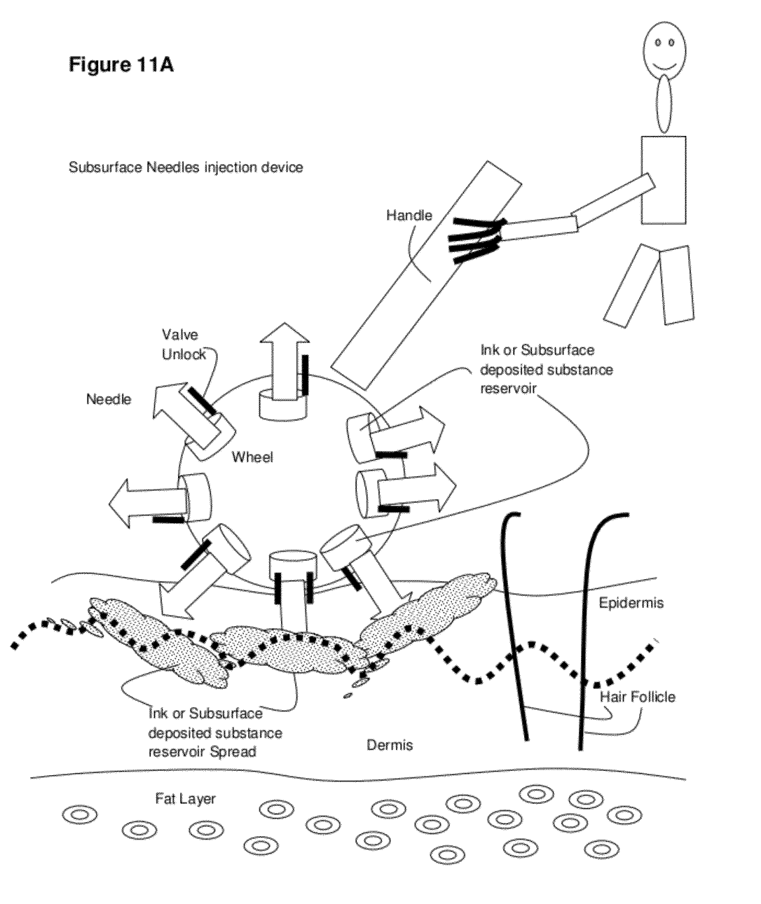

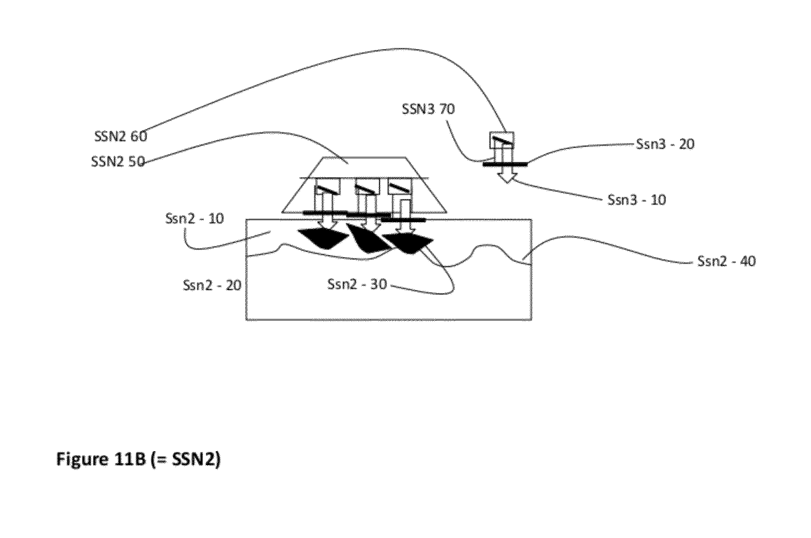

FIG. 11a shows an embodiment of a device creating subsurface skin protection against external influences.

FIG. 11b shows the details of the microinjection embodiments of the device for creating subsurface skin protection against external influences.

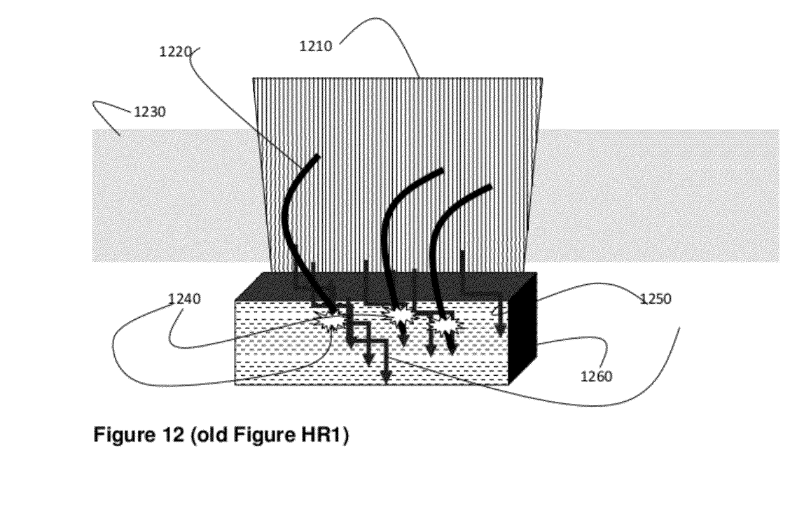

FIG. 12 shows the details of the operation of a pigment absorption of electromagnetic energy hair reduction treatment.

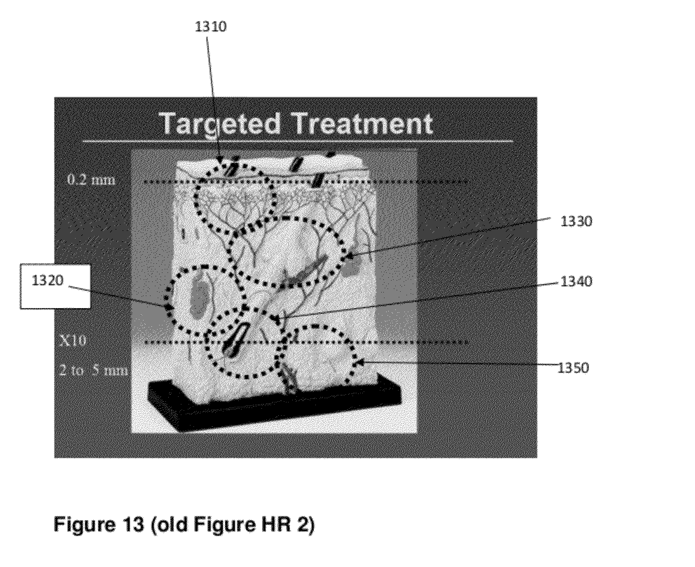

FIG. 13 shows various skin targets that can be treated by the device and method of the present invention.

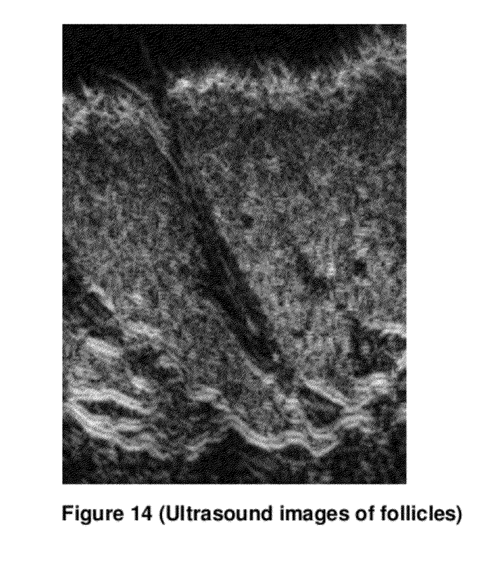

FIG. 14 shows an exemplary capabilities of ultrasound imaging of skin hair follicles.

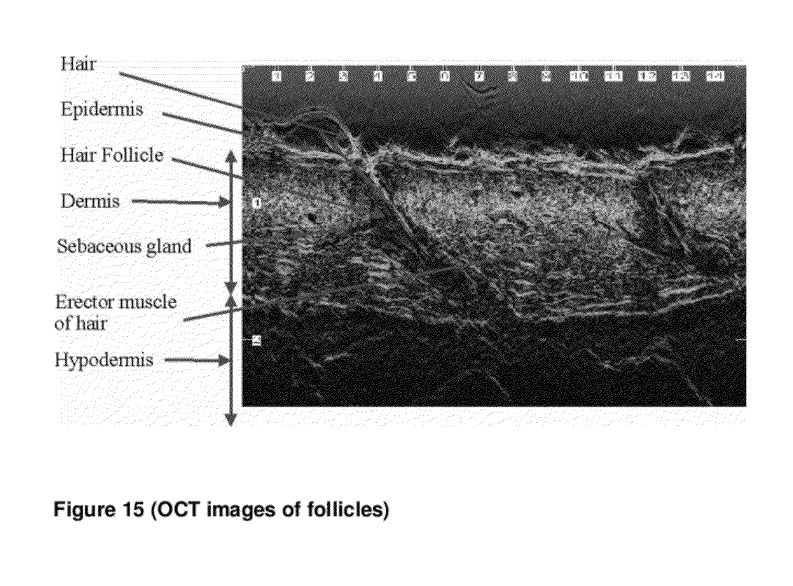

FIG. 15 shows an exemplary capabilities of Optical Coherent Tomography imaging of skin targets including skin hair follicles.

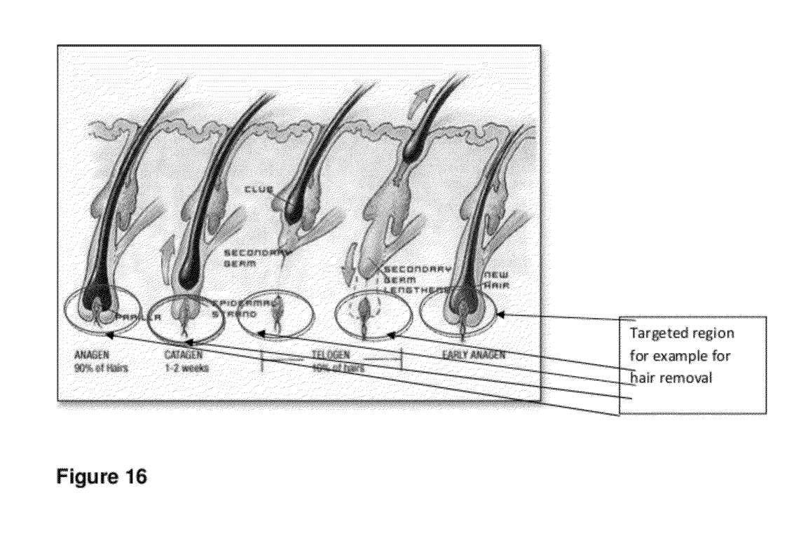

FIG. 16 shows how the present invention can target the hair papilla and the nourishment sources (e.g. blood vessels) of the hair root at all phases of the hair follicle life cycle.

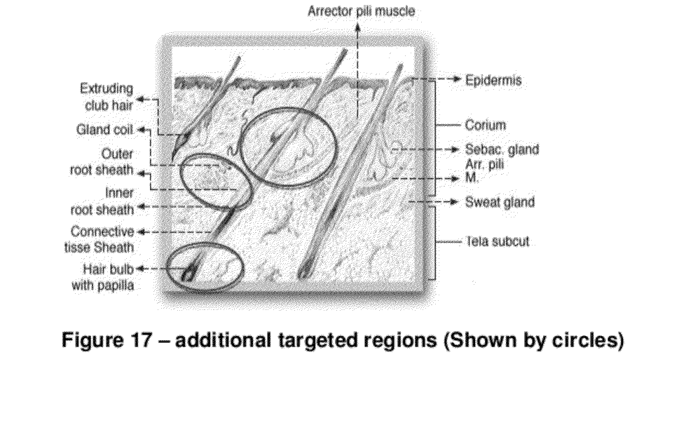

FIG. 17 shows additional targets within the skin of ailment or targets that can be treated with the present invention.

### DETAILED DESCRIPTION OF PREFERRED EMBODIMENTS

Methods for Dermatological Treatment.

First I demonstrate the ability of the present invention to treat subsurface target using characteristics of short pulses.

In one example, we discuss dermatological applications, for example Hair Removal.

In an embodiment of the present invention, an energy source is configured so that its energy is focused in time and space onto a targeted volume under the surface of the targeted material. The interaction with the targeted volume is designed to allow modification or imaging of the targeted medium.

To achieve a desired effect on the targeted medium, a quantity of energy is directed towards the targeted volume of subsurface tissue.

The quantity of energy is concentrated in time and space so that that it reaches an above threshold level of energy density.

Energy density is defined as: (Energy density)=(quantity of energy)/(unit volume)/(unit time) i.e. the volumetric power density is defined as a quantity of energy per time per unit volume and per unit time.

The Energy source preferably produced one or more of said energy types:

Light energy

Electromagnetic (EM) Energy

Mechanical Energy

Sound Energy

Laser Energy

Chemical energy

Thermal energy

Nuclear energy

Ultrasound energy

In one preferred embodiment EM energy, for example, Laser or light energy, is produced by an ultrashort pulse laser source.

1. A package of such energy is caused to propagate from the energy source towards the targeted material. When said package of energy is compressed in time and space so that its volumetric power density is above the threshold of interaction with the targeted tissue to reach a desired effect, the effect will take place at about that region in space within the targeted tissue.

2. A uniqueness and novelty of the described method and device is the ability the ability to modify said EM energy utilizing the following parameters.

In one embodiment, A device for reducing the presence of hair on a skin, the device comprising:

a. a treatment head coupled to a housing;

b. an energy source coupled to a radiative energy source;

c. a controller; and

d. an output port;

e. an imaging member in communications with said controller

In further embodiment the energy source emit pulses of electromagnetic radiation, said pulses are shorter than about 1 ns

In further embodiment the energy source emit pulses of electromagnetic radiation, said pulses are shorter than about 0.1 ns

In further embodiment the energy source emit pulses of electromagnetic radiation, said pulses are shorter than about 10 ps

In further embodiment the emitted pulses are capable of modifying at least some subsurface structures.

In further embodiment the emitted pulses are guided by said imaging system to modify at least one of:

Hair root,

Hair papilla

cell

Tumor cell

Infected cell

Infected tissue

Sebaceous gland

Sweat gland

Blood vessel

Pigmented tissue

In further embodiment the device or method accomplish tis substantially without damaging at least some of the tissue overlying it.

The device of claim seven wherein the targeted tissue is mechanically compressed to reduce electromagnetic radiation scattering.

The device of claim 7 wherein the targeted tissue is deformed to reduce scattering of Electromagnetic radiation.

A device to modify skin against external influences, the device comprises a needle,

a needle,

a reservoir of substance

a member capable of delivering said substance through said needle to the skin

a depth-limiting member, said member limiting the needle penetration to a predetermined depth.

In further embodiment the wherein said external influence is at least one of

Ultraviolent light

Electromagnetic radiation

Heat

Electric energy

Magnetic energy

Chemicals

Poisons

Bacteria

Mechanical impact

Viruses,

Microbial infection

In further embodiment the limiting member limit needle penetration to less than about 5 mm,

In further embodiment the limiting member limit needle penetration to less than about 1 mm

In further embodiment the member limit needle penetration to less than about 0.5 mm

In further embodiment the limiting member limit needle penetration to less than about 0.1 mm

In further embodiment the limiting member limit needle penetration to less than about 50 micrometer

For example, a beam spot size at the targeted volume, wherein said beam is:

Larger than about 1 cm

Larger than about 5 mm

Larger than about 1 mm

Larger than about 0.5 mm

Larger than about 0.1 mm

Larger than about 50 micrometer

Larger than about 25 micrometer

Larger than about 10 micrometer

Larger than about 5 micrometer

Larger than about 1 micrometer

Larger than about 0.5 micrometer

Larger than about 0.2 micrometer

Larger than about 0.1 micrometer

Larger than about 50 nm

Larger than about 25 nm

Larger than about 10 nm

Larger than about 5 nm

Larger than about 1 nm

or

Beam Spot size

Smaller than about 1 cm

Smaller than about 5 mm

Smaller than about 1 mm

Smaller than about 0.5 mm

Smaller than about 0.1 mm

Smaller than about 50 micrometer

Smaller than about 25 micrometer

Smaller than about 10 micrometer

Smaller than about 5 micrometer

Smaller than about 1 micrometer

Smaller than about 0.5 micrometer

Smaller than about 0.2 micrometer

Smaller than about 0.1 micrometer

Smaller than about 50 nm

Smaller than about 25 nm

Smaller than about 10 nm

Smaller than about 5 nm

Smaller than about 1 nm

Preferably the beam spot size is between about 1 um and about 100 um.

More preferably the beam spot size is between about 1 um and about 20 um.

Even more preferably the beam spot size is between about 1 um and about 5 um.

Most preferably the beam spot size is between about 1 um and about 3 um.

In a further embodiment of the present invention, the beam spot size is between about 10 nm and about 1 um and more preferably between about 50 nm and about 500 nm.

In another embodiment of the invention contemplate a series of steps to achieve targeted modification of substances below the target material surface (for example below the surface of the skin or tissue). In the present invention, the operator designs a spot size and pulse energy at the targeted volume (i.e. the operator design a volumetric power density=VPD) of

As a non-limiting example, the inventor will now describe a method and a device for targeting hair bulb and reducing hair growth.

The method is based on the idea that above threshold interaction can be achieved in the targeted zone, for example the region where the hair bulbs are located.

There are two variations for the technique to damage the hair bulb (or hair roots, or hair matrix, or blood vessels feeding the hair bulb).

a) below or at threshold—the ultrashort pulse beam parameters are adjusted so it reaches near above threshold interaction at the vicinity of the hair bulb and rely on the hair bulb higher absorption (due to the present of melanin to reach the needed above-threshold distractive interaction level of energy density.

b) Imaging the region of the papilla and roots. The hair papilla and roots can be located with imaging devices such as Optical Coherent tomography (OCT) or ultrasound or other imaging methods, and the short pulses of energy are directed to the identified region where the bulbs or roots are located so that the power density is above interaction threshold in that region. The pulses of energy can be directed to that energy by manipulating beam parameters (e.g. spot size, pulse duration/temporal focusing, at the targeted location, wavelength, repetition rate) at the targeted volume are such so that the power density is above interaction threshold.

Detailed Embodiment of a. and b. Above.

A. below or at threshold—

1. The operator set the energy source (for example, ultrashort pulse laser) parameters at or below threshold for interaction. One non-limiting exemplary method of doing this is to configure the directing optics or directing members so that the beam focus or beam minimum spot size, or beam most convergence point in space is Below the desired target region (for example, below the hair bulb) and the pulse energy density at the targeted region is lower than threshold for interaction.

Next, the operator manipulate the member directing and focusing the beam so that the location of the minimum beam spot is raised from below the targeted volume toward the targeted volume. In the process, the power density of the beam AT the targeted volume location is increased. At some point during this process, provided that the beam has sufficient volumetric power density to accede the interaction volumetric power density, the beam will damage the and/or irreversibly modify the targeted volume.

The method and device can rely on beam parameters such that even at the minimum spot locations within the targeted material, volumetric power densities (VPD) are ALWAYS below the interaction threshold VPD for the targeted material (e.g. dermis, epidermis, fat tissue, etc.) EXCEPT for targeted locations such as hair bulb and/ or hair follicles (where, for example, the presence of melanin increase the beam energy absorption compared to beam energy absorption in the rest of the host material, for example, the dermis.

Subsequently, the operator decreases the beam spot size at the target by modifying the focal spot position (for example, by raising the focal spot position.

3. A tissue—modifying interaction then occurs when the spot gets small enough to increase the VPD at the targeted volume or targeted spot, to above interaction threshold.

Some of the benefits of the above method are:

No need for guidance

Lower cost of the device

Faster treatment

(among other)

B. Guided fs interaction.

In another embodiment of the present invention, the inventor contemplates a device and a method that is guided by an imaging device or method.

This can be understood with the help of figure USHR1.

An energy source R110 generating a pulse R112, at least one pulse modifier, 116 directing said pulse towards the target, said target is identified by an imaging device, 118. Said imaging device is in communication with said energy source. Said imaging device R118 can identify target location, and additionally or optionally can also identify a modification 119 caused by said interaction at a target location 120.

The imaging member is in communication R125 with the energy source and the energy source and its controllers R127, and can provide feedback to the said energy source and said modifying member so that said interaction location can be moved in space to the desired location.

Figure USHR2 illustrates some of the advantages of the present invention in the removal of hair using guidance or imaging (B). The figure shows the hair structure, location in the skin, and phases. There are four phases in the life cycle of hair:

For example, as described by: homeremediesforhair on Feb. 10, 2011 Under: Hair Knowledge |

The Hair Growth Cycle is devided into 4 Stages of Hair Growth:

Anagen Stage

This is the first phase of the hair growth cycle. It is also known as the active growth phase. In this phase of the hair growth cycle , your hair is growing continuously and consistently for 2-6 years. It begins in the papilla. The growth rate for your hair at this stage is about half an inch per month, however, do note that the span at which the hair remains during this stage of growth is dictated by the genes. Your hair at this point of time will look nourished and thick, and the longer your hair stays in this stage, the faster and longer it will grow. One thing to note is that permanent hair removal can only occur during this active growth phase.

Categen Stage

The second phase of the hair growth cycle is known as the Categen Stage. Also known as the transition phase, the follicle renews itself. Due to disintegration, the hair follicle shrinks and the papilla detaches from the follicle. The hair is then detached from the blood supply. Generally, the follicle will shrink to about 1/6 of its size, causing the hair shaft to be pushed upwards.

About 2-3% of your hair will be in this phase, which lasts for 1-2 weeks.

Telogen Stage

Telogen Stage is the third phase of the hair growth cycle. Otherwise known as the resting phase, the hair follicle is dormant for 5-6 weeks. About 10-15% of the hair will be in this phase.

Return to Anagen Stage

This is the final phase of the hair growth cycle. In this state, hair loss occurs as the preceding hair strand gets pushed up and out by the new hair strand. The dermal papilla moves upward and meets the hair follicle once again, forming new hair in the process. The phase is then cycled back to the Anagen Stage”.

Since modern light and laser based hair removal are based on melanin absorption in the papilla these known in the art methods do not work very well during the tologen phase and categen stage. In addition in, another severe problem of the present known in the art methods based on laser and light devices, is the fact that blonde, gray, white, red, and brown hair do not absorb the light or laser energy well. Thus the melanin in these types of hair is not very effective in capturing and transferring the light or laser energy to the papila and damaging the papila.

By contrast, the method of the present invention cirucumvent this defficieincy by delivering subsurface energy to the dermal papilla region through multiphoton absorption of the ultrashort pulses. The USP interaction is monitored through imaging members, (for example, Ultrasound, OCT, second harmonic, third harmonic, or other multihamronic, flourcense imaging methods or other methods, or other imaging method). The region of the hair bulb is located with imaging devices such as Optical Coherent tomography (OCT) or ultrasound or other imaging methods, and the short pulses of energy are directed to the identified region where the bulbs or roots are located so that the power density is above interaction threshold in that region. The pulses of energy can be directed to the desired region, for example, the hair dermal papilla, regardless of the phases of the hair growth. The energy pulses are directed by manipulating beam parameters (e.g. focal spot location, spot size, pulse duration/temporal focusing, at the targeted location, wavelength, repetition rate, as well as other beam parameters) so that the volumetric power density at the targeted volume, for example, papilla, are above the interaction threshold.

Figure USHR3 shows an exemplary OCT imaging of hair follicles. The figure shows the ability of OCT imaging to locate the hair follicle, hair shaft, hair papilla, dermis, and sebaceous gland among other components of the skin.

Figure USHR 4 shows an exemplary Ultrasound imaging of the hair follicle.

Using an imaging method such as the exemplary OCT or the exemplary Ultrasound imaging, one can guide the interaction to ablate, coagulate, modify or otherwise change the target volume so that at least some of the hair papilla are damaged and the at least some hair growth is prevented or mitigated or reduced.

Some of the advantages of the invention in treating subsurface targets, for example, hair papilla, sebaceous gland, sweat gland or other subsurface targets, are:

Substantially reduced or even eliminated pain (for example, due to reduction in per pulse energy use and minimization of thermal energy deposition).

Substantially reduced or even eliminated collateral damage to tissue outside the targeted volume.

Rapid Operation

The invention, allow for the use of high and very high pulse repetition rates, for example, up to about 1000 Hz, up to about 10 KHz, up to about 100 KHz, up to about 1 MHz, and even up to about 10 MHz, up to about 100 MHz or even up to 1 GHz. A higher rep rate, for example up to a MHz can be generated with sufficient per pulse energy so that modification of the targeted volume which allow the intent of the treated to be achieved (for example, reduction of hair growth, or reduction in the number of sebaceous gland), can still be achieved even with the per pulse energy generating capabilities of a very high pulse rep rate systems described above (for example, ultrashort pulse Ti:Sapph laser at 800 nm and a microjoule of pulse energy, and a pulse rep rate of up to 350 KHz). The high Pulse rep rate, for example, up to about 300 KHz, up to about 1 MHz, up to 10 MHz, or even up to 100 MHz, will allow faster interaction, as photodisruption or material modification locations are rapidly being generated.

Tissue and Hair Modification Modes:

The invention contemplates tow methods for modifying the targeted Tissue (for example the hair roots and hair removal, or sebaceous gland, or sweat glands, or blood vessels, or other tissue targets).

One is through the photodisruption, ablation or other volumetric energy densities above the multiphoton (MP) ablation threshold.

The second is through three dimensional heating and the creation of heat due to accumulation of heat from a pulse train and through MP absorption of each pulse.

As a non-limiting example, we describe an emobidment for such three-dimensional heating comprising an ultrashort pulse source where pulse duration is capapble of reaching volumetric Power Density (VPD) high enough for absorption once a threshold VPD is reached so that absorption within said tissue or target volume is reached. Pulses are then repeatedly heating the target and depending on the total amount of pulses heating the targeted volume per unit time, heat is accumulated and the temperature spatial and temporal distribution within the targeted volume rises.

An exemplary Ti:Saph or Er:Glass lasing medium, or other lasing media with broad band emission, can be used to generate short and ultrashort pulses known in the art (for example, pulse shorter than about a ns, or pulses shorter than about 100 ps, or pulses shorter than about 10 ps) so that said pulses can generate MP absorption at the targeted volume Such pulsed system, can have pulse repetition rate emitted at 1 GHz, 500 MHz, or 100 MHz, or 10 MHz or a MHz, or 500 KHz, or about a 100 KHz, or about 50 KHz or even lower than about 50 KHz.

More preferably such systems can have pulse repetition rate of emission of about 1 KHz. to about 100 MHz, or even more preferably, a rep rate of emission of about 10 KHz to about 50 MHz.

d. Multiple Treatments is possible with less trauma or injury in each treatment and in the overall duration of the sum of all treatments.

e. All tissue types and all hair colors can be treated.

f. A more complete and thorough treatment is possible with more higher efficacy.

Principle of Operation: A device to remove tattoos.

The device comprises of the following components:

An pulsed energy source.

Pulse duration of pulses is less than about 10 ps

Pulse Energy lower than 1 millijoule per pulse

Pulse Energy lower than 100 microjoule per pulse

Energy lower than 0.01 mJ/pulse

Energy lower than 0.006 mJ per pulse

The energy source pulse repetition rate (Pulse Repetition Rate=PRR) of about 0.1 Hz or more

PRR of 1 Hz or more

PRR of 10 Hz or more

PRR of 100 Hz or more

PRR of KHz or more

PRR of about 10 KHz or more

PRR of about 30 KHz or more

PRR of about 50 KHz or more

PRR of about 100 KHz or more

Improve Skin Look

A method for treating skin and improving the look of the skin comprising

ultrashort pulse of energy (below 10 ps in duration) said pulse energy is

such that said energy is below the threshold level to bring any water

element absorbing said energy to above 100 0C.

An embodiment for the method:

1. below or at threshold

2. and raise Focus

3. No pain

4. No Collateral

5. Rapid

6. Multiple Tx

7. All Color

8. More complete

A device to remove tattoo

Pulses shorter than about 10 ps

Pulse Energy:

Energy lower than about 1 mJ per pulse

Energy lower than about 100 microjoule per pulse

Energy lower than about 0.01 mJ/pulse

Energy lower than about 0.006 mJ per pulse

PULSE Repetition Rates:

Pulse Repetition Rate (PRR) of about 0.1 Hz or more

PRR of about 1 Hz or more

PRR of about 10 Hz or more

PRR of about 100 Hz or more

PRR of about KHz or more

PRR of about 10 KHz or more

PRR of about 30 KHz or more

PRR of about 50 KHz or more

To create an interaction zone smaller than the diffraction limit and as small as a few nanometer. In fact, if absorbing elements such as (for example) nanoparticles are used to serve as initiators for the creation of Plasmon or otherwise initiate tissue or other material-modifying interaction, then interaction threshold substantially lower than the tissue or a material native threshold interaction can be reached and the volume of said tissue-modification or material-modification can be significantly lower than the native tissue or material volumes modified. For example such modified tissue or material volume can have diameters of a few nanometers.

EM energy will penetrate materials (or tissue) to varying degrees deepening on the type of materials the EM energy has to transverse on its way to the targeted volume. For example, if the targeted volume is covered with a metal layers, electrons in the metal will generate a shield field that will substantially exclude the radiation from the interior volume protected by the metal.

On the other hand, if the targeted volume is coated by a very thin dielectric layer, for example a thin layer of glass that is substantially non absorbing (for example a total reflection of about 8% to 10 percent of the incoming radiation) then most of the EM energy will arrive at the targeted volume.

In many cases of subsurface interaction (material modification, material imaging, or for diagnostic purposes) the tissue or material transmission, absorption, or scattering is dependent on the EM energy wavelength, power densities as a function of time and space as the EM energy propagates towards the target, and on the material that has to be transverse properties (e.g. the materials absorption, scattering, structure, and composition, as a function of time and space and at the wavelength and beam properties of the propagating EM energy).

In an embodiment of the present invention a package of energy is caused to be able to interact with targeted volume within a depth of from about 0 mm from the surface of the targeted material, for example, targeted mammalian body, to as deep as 10 cm below said targeted surface.

Alternatively, in another embodiment of the present invention a package of energy is caused to be able to interact with targeted volume within a depth of from about 10 micrometer from the surface of the targeted material, for example, targeted mammalian body, to as deep as about 20 cm below said targeted surface.

Alternatively, in another embodiment of the present invention a package of energy is caused to be able to interact with targeted volume within a depth of from about 10 micrometer from the surface of the targeted material, for example, targeted mammalian body, to as deep as about 15 cm below said targeted surface.

Alternatively, in another embodiment of the present invention a package of energy is caused to be able to interact with targeted volume within a depth of from about 10 micrometer from the surface of the targeted material, for example, targeted mammalian body, to as deep as about 10 cm below said targeted surface.

Alternatively, in another embodiment of the present invention a package of energy is caused to be able to interact with targeted volume within a depth of from about 10 micrometer from the surface of the targeted material, for example, targeted mammalian body, to as deep as about 7 cm below said targeted surface.

Alternatively, in another embodiment of the present invention a package of energy is caused to be able to interact with targeted volume within a depth of from about 10 micrometer from the surface of the targeted material, for example, targeted mammalian body, to as deep as about 5 cm below said targeted surface.

Alternatively, in another embodiment of the present invention a package of energy is caused to be able to interact with targeted volume within a depth of from about 10 micrometer from the surface of the targeted material, for example, targeted mammalian body, to as deep as about 2 cm below said targeted surface.

Alternatively, in another embodiment of the present invention a package of energy is caused to be able to interact with targeted volume within a depth of from about 10 micrometer from the surface of the targeted material, for example, targeted mammalian body, to as deep as about 1.5 cm below said targeted surface.

Alternatively, in another embodiment of the present invention a package of energy is caused to be able to interact with targeted volume within a depth of from about 10 micrometer from the surface of the targeted material, for example, targeted mammalian body, to as deep as about 7 mm below said targeted surface.

Alternatively, in another embodiment of the present invention a package of energy is caused to be able to interact with targeted volume within a depth of from about 10 micrometer from the surface of the targeted material, for example, targeted mammalian body, to as deep as about 5 mm below said targeted surface.

Alternatively, in another embodiment of the present invention a package of energy is caused to be able to interact with targeted volume within a depth of from about 10 micrometer from the surface of the targeted material, for example, targeted mammalian body, to as deep as about 3 mm below said targeted surface.

Alternatively, in another embodiment of the present invention a package of energy is caused to be able to interact with targeted volume within a depth of from about 10 micrometer from the surface of the targeted material, for example, targeted mammalian body, to as deep as about 2 mm below said targeted surface.

In an embodiment of the present invention, the inventor has envisioned several methods and devices to enhance the energy penetration into the material or tissue and propagation towards the target material or tissue volume.

For example, means for removing at least some of the liquid or fluid from the targeted tissue or material can be employed. Such means can be, for example, mechanical compression or chemical means for removing energy scattering-causing elements

In yet another embodiment of the present invention, the device and/or method include a member or means for reducing the volume of the targeted material or the volume of the targeted tissue. Such means can be, for example, mechanical compression of the targeted tissue or targeted material, or chemical means for removing scattering elements or scattering components or absorbing components in the tissue so that the light energy or EM energy or other type of energy can better penetrate the tissue or targeted material.

For example, means for removing at least some of the intervening material between the targeted tissue volume or material volume can be employed. Such means can be the Ultrashort pulse laser itself OR the EM energy quanta itself, that can be used to remove, or ablate, or vaporize, or “bore” or “tunnel” or “dig” a subsurface tunnel or voids in the space intervening between the subsurface targeted volume and the surface of the targeted material. I.e. such a method or a device can be used to Vacate, or evacuate, or empty, at least some of the material or tissue (and at least on a temporary basis, i.e. for a limited amount of time) so that at least some of the material is removed or compressed or altered, or modified so that the propagating energy experience less Scattering and/or less absorption as it propagate towards the scattered volume.

For example the volume of the targeted material or volume of the targeted tissue or material can be employed. Such means can be, for example, mechanical compression or chemical means for removing energy scattering-causing elements

Compression: Physical/Mechanical Compression, Optical Compression

In an embodiment of the present invention, an exemplary source of Ultrashort Pulsed EM energy generate a beam with sufficiently large spectral content, as shown in FIG. 1. The energy source can be, for example, a short pulse oscillator 110, with an amplifier 120 and a beam modification member 140. The controller 130 controls the operation and manages input and output control signals including feedback, programming or automation. A beam modifier 140 may optionally include one or more member from the group including: lenses, mirrors, scanners, prisms, or diffraction grating and other diffractive optics elements. The beam modifier may also optionally include a pulse stretcher to stretch the pulse as is known in the art. Optionally, a pulse compressor 150 with members known in the art is used to recompress the pulse as it redirect its components towards the targeted material volume or tissue. A second beam modifier 155 spatially modifies and redirects the optical energy towards the beam coupler. Optionally A beam coupler 160 allows, for example, index matching, reduction of scattering, and enhancement of energy penetration into the targeted material or tissue.

As shown in FIG. 1 the pulse can be stretched when it comes out of the amplifier. It is the amplified and modified by the beam modifier 140. The energy pulse is then redirected by the directing member 144 and as it passes through a pulse compressor the pulse frequency components are manipulated so that the frequency compoenents are rearranged in time and space so that at about the targeted location, said frequency compoentnt of the pulse create a minimum in the pulse duration. Since the pulse volumetric power density Pvm is equal to:

Pvm=Ep/(Va*Tpt) (1)

Where Ep is the energy of the pulse, Va is the volume of target material where the substantially the bulk of the Pulse energy is deposited, and Tpt is the Pulse Duration AT substantially at said absorbing targeted volume.

Since as is shown in equation 1, the Volumetric power density Pvm substantially around the targeted volume is Inversly proportional to the Pulse duration substantially around the targeted volume, Va, as Tpt become smaller, due to the compressing action of the pulses passing through the compressor 150, the Volumetric power density Pvm increases due to the shortening of Tpt and the compressing of the pulse.

As a result, if, for example, the threshold for targeted material modification is Pmmt, by compressing the pulse time duration around the targeted material, according to Eq. 1, the volume Va can become larger and material modification will still occur as the shortening of the pulse (which may also be referred to herein as temporal focusing, or pulse compression) compensate for, for example, the use of larger targeted volume, Vm, or larger spot size Am, or larger focal depth, Zm or both larger Am and Zm.

(note that if, for example, the absorbing volume is a simple cube Vm is simply

Vm=Am*Zm. If the absorbing volume has a more complicated shape or irregular shape then a more complex mathematical/geometrical expression will be used to describe it).

The point is that due to the fact that the pulse duration Tpt around the targeted volume can be compressed, a larger volume at the targeted material location can be used. Such allowed increase in pulse spatial extent as it travels through intervening medium and through the material, allow the avoidance of premature ablation or premature heating, or other premature material or tissue effects, for example, white light generation, self focusing, thermal lensing or other non linear or intensity dependent effects.

These above mentioned effects can be avoided because less spatial focusing, or even substantially no spatial focusing, are used in delivering the energy pulses to the targeted region vicinity, and the operator or user, or the device, uses the Temporal focusing or pulse compression to bring the power energy density at about the region of the targeted volume to a power density level that is substantially above volumetric power density threshold for material or tissue modification. (again, said modification can be thermal, ablative, photo-disruptive, evaporative, explosive, acoustic, mechanical, chemical or other forms of tissue or material modification).

Note: whenever the inventor discusses tissue as a target material it is to be construed as ANY type of physical to be modified. Such physical material may or may not be tissue and may or may not be organic. Similarly, whenever the inventor discusses material as a target material it is to be construed as ANY type of physical material and/ or tissue to be modified. Such material may or may not be tissue and may or may not be organic.

An additional advantage of the present invention is the ability of the method and device contemplated by the present invention to employee a beam of a variety of spot sizes.

For example, exemplary Parameters for the Beam Spot size:

Wherein the beam spot size is Larger than about 1 cm but smaller than about 10 cm

Larger than about 5 mm but smaller than about 10 mm

Larger than about 1 mm but smaller than about 5 mm

Larger than about 0.5 mm but smaller than about 1 mm

More preferably yet, larger than about 0.2 mm but smaller than about 0.5 mm

More preferably, Larger than about 0.1 mm but smaller than about 0.2 mm

Preferably, Larger than about 50 micrometer but smaller than about 100 micrometer

Larger than about 25 micrometer but smaller than about 50 micrometer

Larger than about 10 micrometer but smaller than about 25 micrometer

Larger than about 5 micrometer but smaller than about 10 micrometer

Larger than about 1 micrometer but smaller than about 5 micrometer

Larger than about 0.5 micrometer but smaller than about 1 micrometer

Larger than about 0.2 micrometer but smaller than about 0.5 micrometer

Larger than about 0.1 micrometer but smaller than about 0.2 micrometer

Larger than about 50 nm but smaller than about 100 nm

Larger than about 25 nm smaller than about 50 nm

Larger than about 10 nm smaller than about 25 nm

Larger than about 5 nm smaller than about 10 nm

Larger than about 1 nm smaller than about 5 nm

A broad beam may also be used with temporal pulse compression, wherein said broad beam with said pulse temporal compression allows deeper penetration, said deeper penetration is substantially more free of non-linear effects and substantially more free of self focusing and white light generation.

The improved working parameters with the above mentioned broader or wider beam allows improved work within:

In the eye

In cornea treatment such as LASIK and removal of lens in treatment of cataract.

In the environment of the eye, it is often needed to treat targets that are deep within the eye ball, in the cornea, in the lens, in the sclera, in the retina, floating bodies in the liquid humor, or other targets. In such cases, high power density pulses (for example, for femtosecond or picosecond lasers) can create non linear effect in the eye as they travel towards the targeted area. For example, white light generation, thermal lensing, self focusing or other non linear effects. The compression methods, devices, and methods described above allow the user or doctor, ophthalmologist, to avoid such treatment problems and reach targets deep inside the targeted regions of the eye ball or other targeted materials.

Replacement of Botox injection or in conjunction with Botox injection (for example, creating a subsurface storage space for Botox fluid injection so that said botox application is more evenly distributed and more evenly controlled. Also said Botox fluid release rate is more accurately controlled.

PPA—Principle of operation: Treatment of Skull ailments, imaging and diagnostic of skull ailments.

The invention describes a method and a device for enhancement of delivery of light into body for example, into the brain tissue.

The method employs an ultrashort pulse laser capable of creating a subsurface interaction with tissue.

The interaction is capable of removing at least some of the tissue below the surface of the skin to thin out layers within the volume of the bone.

For example, as shown in FIG. 2, a volume 210 within the frontal bone of the skull, 215, is ablated or vaporized. Said volume is under the surface of the skull 220.

FIG. 3 shows another embodiment of the Skull with subsurface volumes or cavity creation wherein voids, cavities, or spaces of different shapes 330, 335, 340 and 345 are created under the surface of the skull or surface of the skin covering the skull 350, to facilitate penetration of light energy through the skull bone 365 to the brain 360.

In one embodiment the interaction modifies the skull bone sufficiently to enhance transmission by more than about 5%.

In one embodiment the interaction modifies the skull bone sufficiently to enhance transmission by more than about 10%.

In one embodiment the interaction modifies the skull bone sufficiently to enhance transmission by more than about 15%.

In one embodiment the interaction modifies the skull bone sufficiently to enhance transmission by more than about 20%.

In one embodiment the interaction modifies the skull bone sufficiently to enhance transmission by more than about 25%.

In one embodiment the interaction modifies the skull bone sufficiently to enhance transmission by more than about 30%.

In one embodiment the interaction modifies the skull bone sufficiently to enhance transmission by more than about 35%.

In one embodiment the interaction modifies the skull bone sufficiently to enhance transmission by more than about 40%.

In one embodiment the interaction modifies the skull bone sufficiently to enhance transmission by more than about 45%.

In one embodiment the interaction modifies the skull bone sufficiently to enhance transmission by more than about 50%.

In one embodiment the interaction modifies the skull bone sufficiently to enhance transmission by more than about 55%.

In one embodiment the interaction modifies the skull bone sufficiently to enhance transmission by more than about 60%.

In one embodiment the interaction modifies the skull bone sufficiently to enhance transmission by more than about 65%.

In one embodiment the interaction modifies the skull bone sufficiently to enhance transmission by more than about 70%.

In one embodiment the interaction modifies the skull bone sufficiently to enhance transmission by more than about 75%.

In one embodiment the interaction modifies the skull bone sufficiently to enhance transmission by more than about 80%.

In one embodiment the interaction modifies the skull bone sufficiently to enhance transmission by more than about 85%.

In one embodiment the interaction modifies the skull bone sufficiently to enhance transmission by more than about 90%.

In one embodiment the interaction modifies the skull bone sufficiently to enhance transmission by more than about 95%.

In another embodiment a device comprises an energy source, means for directing said energy to a targeted volume under the surface of the human body (for example the skull)

Means for spatially and/or temporally concentrating the energy at a targeted volume under the surface of the body so that said concentrated energy is capable of changing the optical property of said targeted volume of tissue.

The device of claim 1 wherein said energy source is an ultrashort pulse laser.

The device of claim 1 wherein said pulse is spatially focused under the surface of the targeted tissue.

The device of claim 1 wherein said pulse is temporally focused under the surface of said targeted tissue.

The device of claim 1 wherein said pulse is both spatially and temporally focused under the surface of said targeted tissue.

The invention can be further understood with the help of FIG. 3 and FIG. 4.

FIG. 4, shown an anatomical representation of the brain and its components and FIG. 4 shows the skull. The frontal lobe 410 is shown in FIG. 4.

The Frontal Lobe of the cerebrum contains the motor cortex and is associated with muscle movement and parts of speech.

There are three possible ways to define the prefrontal cortex:

as the granular frontal cortex

as the projection zone of the mediodorsal nucleus of the thalamus

as that part of the frontal cortex whose electrical stimulation does not evoke movements

The prefrontal cortex (PFC) is the anterior part of the frontal lobes of the brain, lying in front of the motor and premotor areas.

This brain region has been implicated in planning complex cognitive behaviors, personality expression, decision making and moderating correct social behavior. The basic activity of this brain region is considered to be orchestration of thoughts and actions in accordance with internal goals.

The most typical psychological term for functions carried out by the prefrontal cortex area is executive function. Executive function relates to abilities to differentiate among conflicting thoughts, determine good and bad, better and best, same and different, future consequences of current activities, working toward a defined goal, prediction of outcomes, expectation based on actions, and social “control” (the ability to suppress urges that, if not suppressed, could lead to socially-unacceptable outcomes).

Many authors have indicated an integral link between a person's personality and the functions of the prefrontal cortex.

In an embodiment of the present invention, a device and a method is directed towards delivering a package of energy with sufficient power density (Again, Power Density is defined herein as a quanta of power per unit volume, or energy quanta per unit volume per unit time) to allow activation or modulation, or modification of brain activity.

The challenge to achieve this goal has been the presence of overlying tissue such as hair, skin, muscle, bone, blood, fluids and liquids, or overlying brain tissue.

The present invention method and devices overcome these challenges by utilizing one or more of the following principles of operation:

High power density pulses, for example, pulses from an ultrashort pulse lasers such as Ti:Sapph femtosecond lasers,

Selection of laser parameters, for example, wavelengths that minimize energy absorption by the overlying tissue.

Tissue compression, for example, protruding guards or suction power that compress the tissue, drive out fluids and liquids, and compress tissue components into a more dense form while optionally minimize optical index mismatching at boundaries.

Energy pulse temporal and spatial compression. For example through the use of optical elements such as lenses, prisms, diffraction gratings, mirrors, reflection gratings, etc. for example, through the use of optical focusing, or, for example, through the use of temporal pulse compression so that the pulse duration shrink as it propagates towards the targeted volume.

In a further embodiment of the present invention, as shown in FIG. 3, additionally or optionally, the brain or tissue modifying energy can be introduced through thinner portion of the skull bones, for example, under the roof of the eye, or through the temporal bone.

Additionally or optionally, in a further embodiment of the present invention, as sown in FIG. 3 the energy can be introduced through subsurface “ports” in the skull bone and tissue. Here, for example, through the use of the present invention embodiment described elsewhere herein, of the ability to deliver temporally and spatially focused pulses of energy such that said spatially and/or temporally focused pulses are able to ablate or photo-disrupt or otherwise, remove at least part of the tissue or bone in the skull underneath the surface of the tissue. In such a manner, the present invention proposes removing at least some of the tissue filling the skull and creating subsurface voids that allow enhanced energy penetration into the skull interior and interaction with said brain tissue.

The advantages of such subsurface “ports” is that while enhancing the penetration of energy or light, or for that matter even medication or nutrient or other desired chemicals, substances, or energy forms, into the interior of the skull, the integrity of the surface of the of the skull, and overlying skin, tissue and bone, is maintained, as well as the integrity of a barrier layer that is left posterior (i.e. toward the “voids” or cavities, 330 that are closer to the inner part of the skull between the Brain soft tissue (e.g. white and grey matter) and the “voids” or cavities or “subsurface windows” (SSW) 330, contemplated by the present invention.

Additionally or optionally, the voids, or cavities, or conduits, 330, thus “drilled” or ablated or vaporized, subsurface to allow enhanced optical energy or other form of energy, or substance or chemical, delivery into the brain or into the interior of the skull, can ALSO be filled with low absorption fluid, or liquids, that stabilize and maintain at least some beneficial properties of the skull yet allow enhanced deliver of external energy or substances or products. For example, such “filler” substances, 340, can for example, comprise a clear or low absorption Gels with at least some chemical or substances that retard, or discourage bone growth, for example said fillers, 340 discourage tissue or bone growth.

Additionally or optionally, the voids, or cavities, or conduits, 330, thus “drilled” or ablated or vaporized, subsurface to allow enhanced optical energy or other form of energy, or substance or chemical, delivery into the brain or into the interior of the skull, can ALSO be filled with low absorption fluid, or liquids, that prevents or slow down refilling or changes of the voids or cavities or conduits 330.

The creation of such Subsurface voids or subsurface windows (SSW) 330, can be made with the lasers parameters and pulse temporal focusing or pulse compression techniques described above and in related application.

The SSW can be, for example, less than about,

Less than about 1 micrometer in diameter.

Less than about 5 micrometer in diameter

Less than about 5 micrometer in diameter

Less than about 19 micrometer in diameter

Less than about 15 micrometer in diameter

Less than about 25 micrometer in diameter

Less than about 35 micrometer in diameter

Less than about 50 micrometer in diameter

Less than about 75 micrometer in diameter

Less than about 100 micrometer in diameter

Less than about 125 micrometer in diameter

Less than about 150 micrometer in diameter

Less than about 175 micrometer in diameter

Less than about 200 micrometer in diameter

Less than about 250 micrometer in diameter

Less than about 350 micrometer in diameter

Less than about 500 micrometer in diameter

Less than about 750 micrometer in diameter

Less than about 1000 micrometer in diameter

Less than about 1250 micrometer in diameter

Less than about 1500 micrometer in diameter

Less than about 2 mm in diameter

Less than about 4 mm in diameter

Less than about 5 mm in diameter

Less than about 7.5 mm in diameter

Less than about 10 mm in diameter

Less than about 12 mm in diameter

Less than about 15 mm in diameter

Less than about 17.5 mm in diameter

Less than about 20 mm in diameter

Less than about 25 mm in diameter

Less than about 30 mm in diameter

Less than about 40 mm in diameter

Less than about 50 mm in diameter

Less than about 55 mm in diameter

Less than about 60 mm in diameter

Less than about 75 mm in diameter

Less than about 100 mm in diameter

Less than about 125 mm in diameter

Less than about 150 mm in diameter

Less than about 200 mm in diameter

The subsurface window can be created, for example, with their antirior surface (i.e. the surface facing the outside of the skull and the external surface of the skin covering the body of the treatment subject, extending

from about 1 micrometer below the surface of the skin

from about 5 micrometer below the surface of the skin

from about 7.5 micrometer below the surface of the skin

from about 10 micrometer below the surface of the skin

from about 15 micrometer below the surface of the skin

from about 20 micrometer below the surface of the skin

from about 25 micrometer below the surface of the skin

from about 37 micrometer below the surface of the skin

from about 50 micrometer below the surface of the skin

from about 75 micrometer below the surface of the skin

from about 100 micrometer below the surface of the skin

from about 150 micrometer below the surface of the skin

from about 200 micrometer below the surface of the skin

from about 250 micrometer below the surface of the skin

from about 350 micrometer below the surface of the skin

from about 400 micrometer below the surface of the skin

from about 500 micrometer below the surface of the skin

from about 600 micrometer below the surface of the skin

from about 750 micrometer below the surface of the skin

from about 1000 micrometer below the surface of the skin

from about 1500 micrometer below the surface of the skin

from about 2000 micrometer below the surface of the skin

from about 3000 micrometer below the surface of the skin

from about 4000 micrometer below the surface of the skin

from about 5000 micrometer below the surface of the skin

from about 7500 micrometer below the surface of the skin

from about 10,000 micrometer below the surface of the skin

More than about 10 mm below the surface of the skin but less than about 30 mm below the surface of the skin.

The height of the SSW (i.e. the extent of the SSW in the dimension directed from the surface of the skin towards the surface of the brain, i.e. towards the position of the targeted volume, or volume targeted for treatment within the skull), can range from about:

about 1 micrometer

about 5 micrometer

about 7.5 micrometer

about 10 micrometer

about 15 micrometer

about 20 micrometer

about 25 micrometer

about 37 micrometer

about 50 micrometer

about 75 micrometer

about 100 micrometer

about 150 micrometer

about 200 micrometer

about 250 micrometer

about 350 micrometer

about 400 micrometer

about 500 micrometer

about 600 micrometer

about 750 micrometer

about 1000 micrometer

about 1.5 mm

about 2 mm

about 3 mm

about 4 mm

about 5 mm

about 6 mm

about 7 mm

about 8 mm

about 9 mm

about 1 cm

about 1.5 cm

about 2 cm

about 2.5 cm

about 3 cm

about 4 cm

about 5 cm

about 6 cm

about 7 cm

about 8 cm

about 9 cm

about 10 cm

more than about 10 cm but less than about 20 cm.

When I say “about” I mean the value (e.g. 1 cm) plus or minus 50% of that value (e.g. if the value is, for example, 1 cm then “about” would mean any value between about 0.5 cm and about 1.5 cm.

The SSW can be made by debulking, vaporizing or otherwise substestnetially removing most of the material within about the volumes described herein above.

OR, additionally and optionally, said SSW 330 can be made by creating a pattern of removed voids or cavities, or smaller conduits 340 within the overall SSW 330. Such pattern of voids 340 within the larger SSW 330 can optionally form a density of (expressed as percent voids 340 volume within the overall SSW volume 330) of about:

About 1%

About 2.5%

About 5%

About 7.5%

About 10%

About 15%

About 20%

About 25%

About 35%

About 40%

About 50%

About 60%

About 70%

About 75%

About 80%

About 90%

About 95%

About 98%

About 99%

About 100%

Principle of Operation: Correcting Vision in the Eye and Treatment of Eye Ailments:

Another embodiment of the present invention is shown in FIG. 5 and FIG. 6. In this embodiment the system and method to modify vision is contemplated. It has been described in the past by the present inventor as well as other prior art, it is known to cut flaps or subsurface lines within the cornea.

The present invention, contemplates creating subsurface structures as described by the parameters tables shown in table 1a to table 1e, in the cornea or lens, or both in the cornea and Lens. Optionally, or additionally such structures can also be cut in sclera or other structures of the eye.

The invention contemplates using such structures to modify the elastic properties or the optical properties or at least one of a group of properties of the eye using such structures 520. Among the group of such properties of the eyes, are shown in table 2.

Table 2:

Elastic properties

Optical properties

Refractive properties

Thermal properties,

Hardness

Opacity

Absorption

Scattering

Electrical properties,

Other properties.

Additional Embodiment for Ophthalmic Applications:

Additionally or optionally, a fluid or liquid is injected to the structures thus created within the Lens or the cornea, or other structures within the eyes. Such fluid or liquid may be injected or otherwise inserted into the eye and its volume, pressure, or density, or other relevant characteristic may be adjusted to allow control (possibly even dynamic, real time, adjustable control) of the curvature of the cornea or lens or other components of the eye, to allow treatment of refractive power of the eye, focusing power of the eye, and/or corrections of such ophthalmic conditions as myopia, presbyopia, astigmatism, cataract, or other ophthalmic conditions.

In Another Embodiment

The energy source, for example a fs laser, creates the storage for a fluid or a liquid.

The Fluid or liquid can, for example, be a memory retaining polymer.

A Fluid containing absorbers for enhanced absorption of the incoming energy, or fluid which is doped with absorbers, for example nanoparticles that can expend upon the delivery of a willfully triggered external signal, for example a laser signal that cause them to expand.

Depending on the position of said fluid pockets, they can either inflate or deflate the lens

for example the crystalline lens of the eye, thus causing increase or decrease in the focusing power of said lens.

For example, an activation of the absorbers in pocket 620 can cause a lens to inflate and focus more. Thus the invention describe a method and a device that allows us to overcome and correct presbyopia.

This is illustrated further with the help of FIG. 6.

One can create micro structure in accordance with the cavities or voids dimensions specified by the present invention.

The present invention contemplates inserting fluids, for example, doped with nanoparticles that respond to external energy and expends and perverse their shape or cool off and retract

Or expend in one part of the cornea to stretch, and expend in another part to contract.

While inactivation of 620, can be achieved, for example, by activation of the absorbers 610,

Thus causing deflation of the lens and pocket 620 allowing flattening of the lens (by “flattening” I mean making the lens flatter in appearance or in curvature, i.e. more oval and flat instead of the lens being more round and more curved).

Lowering of the lenses focusing power, and, for example, treatment of myopia.

So in effect, one trigger—620 causes expansion and bulging.

The second trigger(s) 610 cause flattening including flattening (or turning off}of the first trigger.

The two triggers work like two sets of springs with on and off switch wherein the energy is provided externally by the external energy source (e.g. laser light beam etc.)

One of the embodiments and principles of operation of the present invention thus comprises (as shown in FIG. 6):

1. The Creation of a storage space 620 and 610 (among other storage volumes in different locations and with desired structures). Such storage space can be created with the aid of an external energy source, for example, an external fs laser or USPL and its ability to create subsurface structures in the eye (or in other tissue or body parts, or other materials and substances).

2. The insertion (for example, injection) of a fluid or liquid capable of modifying at least one property of the eye (or other tissue or body part). For example, the injection of substance that can be willfully triggered by a signal from an operator so they expand. For example, a biocompatible fluid or liquid or other substance that can expend upon heating, wherein such biocompatible substance also contains a substance that can absorb a radiation from an external laser (for example, a biocompatible substance containing nanoparticles that converts said external energy into heat), and thus expending and causing the tissue (for example a lens or a cornea) to change it shape.

3. Activation of said inserted substance by an external source, and obtaining a desired shape (and possibly function) of the treated organ. For example, insertion of nanoparticles doped polymers into pockets or voids prepared by an external energy source such as a laser, or ultrashort pulse laser, can allow the user or operator to change the shape of a lens or cornea to improve vision.

4. Such changes to tissue or organs (for example, the eye crystalline lens) can be reversible and/or adjustable as the external source can be willfully used as described herein above to modify or adjust the changes.

Principle of Operation: Bacteria with Florescence or Absorption Filters:

In another embodiment of the present invention a method and a device for detection of bacteria is described.

The device comprises a light source with a spectra emission that covers at least some of the excitation wavelength that causes the targeted bacteria to floursce. The Energy source can be for example, a broad band flash lamp, even a compact broad band flash lamp with a filter that allow light different from the emission wavelength of the bacteria to be emitted.

The excitation energy source can, for example be similar to the one made with a a disposable camera flash lamp and its related circuitry wherein a filter is used to limit the emission from the flash lamp to wavelnths shorter than the one emitted by the bacteria.

The bacterial emission is allowed to pass through a band-pass optical filter that blocks any other stray light. If a bacterial is present, the emission wavelength from the fluorescence bacteria passes to detector, for example a photodiode, and the signal detected then indicate the presence of the bacteria or other pathogen or virus, and can also be correlated to the amount of bacteria or pathogen present.

To avoid errors and increase accuracy, since if a bacteria generate an emission following an excitation, the emissions of Wavelength (WL) no. 1, e.g. WL1 and WL2 have certain proportions or ratio, that is typical to that bacteria. Thus, knowing the ratio will confirm the type of bacteria and will ensure that the WL line that appear is not a stray light effect.

Thus, if the bacteria has TWO emission fluorescence wavelengths, then part of the emitted light can be direct to a band-pass filter that allow only WL1 to be transmitted and a second part is directed to second line filter that allow only the second WL, WL2 to be transferred. Thus ONLY if both WL1 and WL2 are present the device confirms that a bacteria of the type that emits both WL1 and WL2, is actually present, and not an error that happened to have generated one of the WL by an error.

A device for detection and treatment of bacteria is shown in FIG. 7:

An exemplary device for detection and treatment of bacterial may comprise:

A an excitation source (A).

Said excitation source comprises of the following members:

720 power source (e.g. AAA batteries)

725 capacitors,

730 trigger

735 charge button

755 control board microprocessor

750 lamp/flash lamp (Usually broad band from about 300 nm or 400 nm to as much as 1.2 microns in wavelength. For Fluorescence excitation we can filter out the IR and Visible and allow mostly blue to UV excitation—e.g. see example below).

765 a window

B—breath or bacteria input

C—Breath or blow outlet

D—Sample Chamber

E—Lenses

G—Filters/band path filters.

H—Microprocessors.

Example of Bacterial Detection:

One can achieve rapid detection and differentiation of bacteria, e.g. Escherichia coli, Salmonella, and Campylobacter, which are the most commonly identified commensal and pathogenic bacteria in foods, using fluorescence spectroscopy and multivariate analysis.

A most common and urgent need is the identification, detection and destruction of bacteria such as the strep bacteria in human infection, or bacteria causing bad breath.

Fluorescence spectra can be collected over a range of 200-700 nm with 0.5 nm intervals flash lamp Fluorescence detectors as described herein above.

Once an optimum excitation and emission wavelengths for individual

bacteria are identified, the excitation spectra can be generated for example one that shows maximum excitation values at 225 nm and 280 nm and one maximum emission spectra at 335-345 nm. Refinement with Two wavelength emission and multi wavelength excitation as described above can be employed.

3. Needle Sunscreen and subsurface interaction.

FIG. 11 (=Figure SSN1)

Preferred embodiment, subsurface modification and injection of substance, as shown in FIG. 11.

This can create, body art, long term skin protection, or wrinkle removal similar to Botox™.

FIG. 11B. shows how an injectable substance is injected by the needle of the wheel shown in FIG. 11.

### Preferred Embodiment

Cavity Diagnostics:

A micro-cavity biosensor monitors optical resonances in micro- and nanostructures for label-free detection of molecules and their interactions. Recent applications allow optical microcavities for nanoparticle detection, trapping and manipulation, and I will highlight different modalities for ultra-sensitive label-free bio-sensing.

The invention contemplates the creation of microcavities (for example—with fs lasers) within a biological tissue, for example within the environment of the eye.

Changes in light trapped within the eye are then monitored to detect the presence of atoms, molecules, proteins, and viruses within the tested environment. Including insulin level.

Ultrashort pulse lasers has several unique interaction characteristics that make them ideal for several traditional dermatological treatments such as tattoo removal, hair reduction, skin rejuvenation, and treatment of pigmentation.

The characteristics include:

Ability to interact with subsurface structures within the skin without damaging the skin.

Ability to heat subsurface structures within the skin without damaging the surface of the skin.

Ability to target and damage very localized regions of the treated volume with minimal or no collateral damage.

Color-blind interaction (insensitive to tissue type or hair color)

Threshold enabled interaction which can be tuned for tissue or target region absorption enhancement.

Subsurface skin modification (SSM) procedures are based on the physical phenomenon of “3-D optical breakdown for material modifications”. Using this phenomenon, microscopic interaction can be created inside the surface of the skin by focusing ultra short pulses of laser light into it.

In Hair Treatment, (as shown in FIG. 1 below) an Ultrashort pulsed laser beam is directed and focused at the hair follicle or hair bulb elow the surface. The location of the hair bulbs can be determined through the use of OCT or through other imaging and feedback techniques. In essence the Ultrashort pulse laser generates a “subsurface haircut” or a subsurface elimination of the tissue responsible for hair growth. The method is hair-color blind in the sense that the interaction is not very sensitive to hair color. The interaction can create a plane of damage at the level of the papillae or cell matrix feeding the hair or responsible for its growth. The damage is permanent to the hair bulb and matrix but is minimal and recoverable for the rest of the skin tissue. Tissue clearing and tissue compression techniques can also be used to enhanced penetration.

Another substantial advantage of the multi-photons ultrashort pulse based method over conventional method (see FIG. 12) is in hair removal and other skin methods is their ability substantially delivers much of the light to the target as oppose to “wasting” it on collateral damage and adjacent tissue.

As shown in FIG. 12, a beam of light (for example, a broad ban flash lamp pulse, or a CW diode laser beam, 1210) is directed towards the skin epidermis 1230 and Dermis 1260.

The Beam is absorbed by the melanin in the colored hair 1220, and created an interaction that at least partially damages the hair.

The interaction is shown by 1240. The Light photons in the beam scatter around the tissue 1260, for example as in the passes 1250, until they encounter the melanin colored hair and get absorbed.

To create at least partial damage to the hair 1220 and preferably to the hair bulbs and roots.

For example, if an 800 nm light source is use, some absorption and heating occurs as the beam passes through the surface and upper layers of the skin. In fact, most of the beam is not used in the target but rather is scattered, reflected and ultimately (whatever portion is not reflected or scattered out of the target) is absorbed in the tissue regions outside the targets.

The beams are often very large in diameter (e.g. up to 1 inch or even 3 inch) and the interaction is based on melanin absorption in the hair follicles.

For this reason the light has to be absorbed well by the hair, and hence blonde, white, red, and gray hair are not very responsive. In addition because of the hair growth phases, light may be mimimally absorbed during the dormant stage of some hair follicle. Further, depending on the amount of melanin presence absorption may be week. Finally, to compensate, some devices and clinicians may use higher pulse or source energy resulting in burn and collateral damage.

The case is different for short and ultrashort pulse capable of multi-photon interaction, where the light is focused in time and/or space to create an above threshold event at or near the target. When such a threshold power density is created, the much of the pulse energy (except for the energy reflected at the surface or absorbed/scattered on its way to the target) is absorbed by the target or its immediate surrounding and is used to create the photo-disruption damaging event regardless of the absorbing capabilities or color of the hair at the target.

In other words, ONE DOES NOT depends on the target absorption to kill the hair bulb. One kills the hair bulb by aiming and “shooting” the pulse into it.

FIG. 13 shows possible target of the method and device.

Similar to the described short pulse energy Multiphoton (MP) interaction and modification of the hair bulb 1340. The interaction can be directed towards fat or cellulite layers 1350, the interaction can also be directed towards the sebaceous gland 1330, and acne problems, the sweat gland 1320, or towards pigmentation and vascular blemishes 1310, or other vascular problem such as Port wine stains, 1310, hemangiomas, rosacea, cafe Ole' stains, hypopigmentation or hyperpigmentation or other problems in the epidermis and/or dermis.

Alternatively or additionally, the Ultrashort pulse beam can be made to converge (temporally or spatially) towards the targeted hair root. Above the hair root, the pulse energy density is too low for interaction, at the hair root, the pulse energy density is close to interaction threshold but can be made CLOSE to said interaction threshold and thus, when encountering the slightly higher absorption of root the pulse energy density reaches the interaction threshold and causes permanent damage to the hair root or hair too matrix. This version of the USPL interaction does rely on slight absorption but only to trigger the above threshold event. The pulse compression (temporal or spatial) is the mechanism responsible for bringing the energy close to interaction and sparing overlying or underlying tissue. The higher absorption in the roots is the mechanism responsible for sparing the lateral damage to adjacent lateral tissue.

Similar USPL mechanisms can be used in the treatment of sebaceous gland for permanent curing of moderate to severe acne, in treating tattoos, and in treatment of port wine stains, vascular or pigmented lesions. Additionally, similar USPL can be used to treat fungus and other nail ailments.

FIG. 14 and FIG. 15 show the capability of technologies known in the art, for example, ultsound imaging, FIG. 14, or Optical Coherent Tomography (OCT) FIG. 15, to image hair follicle and hair roots and thus guide the short or ultrashort pulse interaction with subsurface capabilities as described by the present invention.

In one embodiment, a device for enhanced energy delivery to the skin is contemplated. The device comprises an energy source, means for directing energy from said energy source towards said target, means for compression of a target material.

In another embodiment, a device for enhanced energy delivery to the skin is contemplated. The device comprises an energy source, means for directing energy from said energy source towards said target, means for mechanical compression of a target material and means for synchronizing said energy source and said mechanical compression mean.

In further elaboration of the above embodiments, the means for compression of the target material comprise a contact surface configured to contact and press against the target material.

Further elaboration of the device above envision using a mechanical means to drive said compressing contact surface into the target material, for example, as a non-limiting example, a motor driver, a shaft and a piston can be used, wherein said motor may be an electric motor or any other kind of motor known in the art.

In further embodiment of the devices above, said mechanical means comprises a surface capable of deforming and exerting mechanical pressure on the target surface.

Additional embodiment of the device further envision the mechanical means comprises a contact surface configured to contact and apply mechanical pressure on a target material, and means to drive said contact surface towards the target surface. The contact surface may be configured one or more of a group comprising:

Flat surface

Surface with pins or protruding members

Curved surface

Rough surface

Surface with pins

Surface with protruding rods

Surface with protruding pyramids

Irregular protrusions form the surface

The device may further comprise of an electric motor as a means for driving said contact surface towards the target material.

The device may further comprise a mechanical motor for driving said contact surface towards the target material.

The device may further comprise of an electric motor as a means for driving said contact surface towards the target material.

The device may further comprise a transducer or an actuator for driving said contact surface towards the target material.

The device may further comprise of an piezo-electric crystal driver as a means for driving said contact surface towards the target material.

The device may further comprise of an other me means known in the art for driving said contact surface towards the target material.

The device above may further comprise a contact surface which may be transparent.

In further embodiment the device contact surface may be made of one or more of the following materials:

Metal

Plastic,

Dielectric

Glass

Semiconductor

Super conductor

Aluminum

Stainless still

Copper

Brass

Silver

Gold

Titanium

Carbon composite

Transparent materials

Biocompatible materials

Opaque materials

Thermally conductive material

Thermally isolating material

Electrically conductive materials

Electrically insulating materials.

In additional embodiment of the device above—the above device contact surface may be cooled by one or more of the following methods:

Thermoelectric cooling

Spray cooling

Freon—type coolants

Air cooling

Expending gas cooling

Circulating liquid cooling

Circulating fluid cooling

Other cooling methods known in the art.

In additional embodiment of the device above—the above device contact surface may be heated by one or more of the following methods:

Thermoelectric heating

Heating using spray

Heating using steam

Warm air heating

Electric heating

Mechanical heating

Circulating warm liquid

Circulating warm fluid

Other heating methods known in the art.

In further embodiment the contact surface may be made partly of metal and partly of dielectric.

In further embodiment the contact surface may be partly heated

In further embodiment the contact surface may be partly cooled

In further embodiment the contact surface may be partly cooled and partly heated

In further embodiment the contact surface may be made at least in part of metal said metal is connected to a cooling source, for example, TEC, or cooling spray, or cooling flow.

I claim:

1. A method for protecting a surface, the method comprises: applying energy to the surface moving said energy source so that a pattern is formed on the surface of said surface so that a pattern is formed on said surface, the surface comprises a texture or a pattern, said pattern or texture comprises a topography or elevated or lowered surface features, the dimension of the elevated or lowered features are on the order of a few micrometers, and the features separating said elevated or lowered features are also on the order of a few micrometer.

2. The method of claim 1 wherein said surface is an organ.

3. The method of claim 1 wherein said surface is a skin.

4. A device for creating a protection of a target surface, the device comprises: providing an energy source, an energy coupler coupling the energy from the source to the surface so that said energy forms a texture or a pattern is formed on the surface of said surface so that the surface acquires a texture or a pattern, said pattern or texture comprises a topography or elevated or lowered surface features, the dimension of the elevated or lowered features are on the order of a few micrometers, and the features separating said elevated or lowered features are also on the order of a few micrometer.

5. The device of claim 4 wherein said pattern comprises by a plurality of spaced apart features attached to or projected into said base article, said plurality of features comprising at least one feature having a substantially different geometry, wherein neighboring patterns share a repeated feature, the plurality of features are spaced apart and wherein said features are having at least one micron-scale dimension.

6. The device of claim 4 wherein the targeted material has a surface; said surface having a topography comprising a pattern defined by a plurality of spaced apart features attached to or projected into said base article, said plurality of features comprising at least one feature having a substantially different geometry, wherein neighboring patterns share a common feature, the plurality of spaced apart features having at least one micron-scale dimension.

7. A device for reducing the presence of hair on a skin, or for modifying other subsurface skin targets, the device comprising:
a treatment head coupled to a housing;
an energy source coupled to a radiative energy source;
a controller; and
an output port;
an imaging member in communications with said controller.

8. The device of claim 7, wherein said energy source emit pulses of electromagnetic radiation, said pulses are shorter than about 1 ns.

9. The device of claim 7, wherein said energy source emit pulses of electromagnetic radiation, said pulses are shorter than about 0.1 ns.

10. The device of claim 7, wherein said energy source emit pulses of electromagnetic radiation, said pulses are shorter than about 10 ps.

11. The device of claim 7, wherein said emitted pulses are capable of modifying at least some subsurface structures.

12. The device of claim 7, wherein said emitted pulses are guided by said imaging system to modify at least one of: Hair root, Hair papilla, Cancer cell, Tumor cell, Infected cell, Infected tissue, Sebaceous gland, Sweat gland, Blood vessel, Pigmented tissue, Substantially without damaging at least some of the tissue overlying it.

13. The device of claim seven wherein the targeted tissue is mechanically compressed to reduce electromagnetic radiation scattering.

14. The device of claim 7 wherein the targeted tissue is deformed to reduce scattering of Electromagnetic radiation.
